(19) United States
(12) Patent Application Publication
(10) Pub. No.: US 2007/0031828 A1
(43) Pub. Date: Feb. 8, 2007

(54) Assay

(75) Inventors: Brigitte Desiree Alberte Colau, Rixensart (BE); Gary Dubin, King of Prussia, PA (US); Marie-Therese Martin, Rixensart (BE); Wim Quint, Delft (NL); Leen-Jan Van Doorn, Delft (NL)

Correspondence Address: GLAXOSMITHKLINE, Corporate Intellectual Property - UW2220, P.O. Box 1539, King of Prussia, PA, 19406-0939, US

(73) Assignee: GlaxoSmithKline Biologicals S.A Delft Diagnostic Laboratory

(21) Appl. No.: 11/581,251

(22) Filed: Oct. 12, 2006

Related U.S. Application Data
(63) Continuation of application No. 10/485,838, filed on Oct. 5, 2004.

(30) Foreign Application Priority Data
Aug. 8, 2001 (EP) 01202991.4

(51) Int. Cl. C12Q 1/68; C12P 19/34; C12Q 1/70

(52) U.S. Cl. 435/5; 435/6; 435/91.2

(57) ABSTRACT

A process for identification of type-specific polynucleotide sequences in a sample, the process comprising the steps of (1) contacting polynucleotides from the sample, or derived from the sample, with a plurality of type-specific probes in the context of a solid support, and detection of any type-specific hybridisation; and (2) contacting polynucleotides in the sample, or derived from the sample, with type-specific primers for those types capable of being detected by the hybridisation step in step 1, but not so detected, in a type-specific amplification reaction.

### CROSS-REFERENCE TO RELATED APPLICATIONS

This application is a continuation of Ser. No. 10/485,838, filed Oct. 5, 2004, which is a 371 application of PCT/EP02/08791, filed on 6 Aug. 2002.

The present invention relates to a method for nucleic acid detection and analysis.

In particular the invention relates to methods for the detection and typing of nucleic acid, e.g. for diagnosis, and identification of mutations in genes associated with disease.

The importance of accurate and comprehensive genotyping analysis is exemplified by the typing of human papillomavirus (HPV). Papillomaviruses are small DNA tumour viruses, which are highly species specific. So far, over 100 individual human papillomavirus (HPV) genotypes have been described. HPVs generally infect either the skin (e.g. HPV-1 and -2) or mucosal surfaces (e.g. HPV-6 and HPV-11) and usually cause benign tumours (warts) that persist for several months or years. Such benign tumours may be distressing for the individuals concerned but tend not to be life threatening, with a few exceptions.

Some HPVs are, however, associated with more serious disease such as cancers. The strongest positive association between an HPV genotype and human cancer exists between HPV-16 and HPV-18 and cervical carcinoma. Individuals with, for example, HPV 16 and/or 18 infection are considered to be at high risk of developing the disease. Cervical cancer is the most common malignancy in developing countries, with about 500,000 new cases occurring in the world each year.

At present large scale nucleic acid typing is generally carried out by probe array hybridisation.

By way of example, in the HPV field, Sahli recently disclosed a method for diagnosis of HPV infection by PCR amplification of HPV DNA followed by probe array hybridisation and DNA sequencing [R. Sahli, WHO meeting Heidelberg, Germany, 6-7 Mar. 2001. “Assessment and Harmonisation of Laboratory Diagnostic procedures related to HPV Vaccine research and Development”]. The method comprises a first PCR step to amplify HPV DNA within a sample using broad spectrum primers specific to HPV in general, but not specific to any HPV type in particular. This is followed by reverse blot hybridisation of the PCR fragments so generated with multiple type-specific oligonucleotide probes bound to a solid support. The hybridisation of a PCR fragment with a type-specific oligonucleotide allows specific HPV types in the sample to be detected. The use of hybridisation techniques in this way allows the screening of a sample against multiple HPV probes after a single PCR, and avoids the need for multiple individual specific PCR reactions.

For those samples identified as HPV-positive by the initial PCR reaction, but for which there is no positive hybridisation reaction with an HPV specific probe, direct sequencing of the DNA is carried out to identify the HPV type, or confirm the presence of a new type. Sahli discloses that HPV-positive but hybridisation-negative amplicons always corresponded to HPV types or subtypes not represented in the hybridisation specific array, as determined by sequencing.

Screens involving a similar hybridisation detection systems are known for identification and typing of a number of bacterial and viral genes, such as those derived from HIV-1, HCV, HBV, Helicobacter pylori and Mycobacteria, mycoplasma, along with typing of genes such as p53, involved in cancer.

The present invention provides an improved method for nucleic acid detection and typing.

In a first aspect, the invention relates to a process for identification of type-specific polynucleotide sequences in a sample, the process comprising the steps of:
(i) contacting polynucleotides from the sample, or derived from the sample, with a plurality of type-specific probes in the context of a solid support, and detection of any type-specific hybridisation; and
(ii) contacting polynucleotides in the sample, or derived from the sample, with type-specific primers for those types capable of being detected by the hybridisation step in step 1, but not so detected, in a type-specific amplification reaction.

In a preferred embodiment the invention relates to a process for identification of type-specific polynucleotide sequences in a sample comprising the steps of
(i) amplification of nucleic acid from the sample using broad spectrum primers;
(ii) contacting polynucleotides obtained from (1) with a plurality of type-specific probes in the context of a solid support, and detection of any type-specific hybridisation; and
(iii) contacting polynucleotides in the sample, or derived from the sample, with type-specific primers for those types not detected by the hybridisation step in step 2, in a type-specific amplification reaction.

Preferably the process additionally comprises a further step 1b, wherein nucleic acid amplified by broad spectrum primers in step 1 is analysed to confirm the presence of general polynucleotide types of interest prior to hybridisation. Only those samples positive for the general polynucleotide types of interest are screened in step 2. Preferably the amplimers obtained from (1) are contacted with a mixture of general probes which are capable of recognising a broad range of types, preferably in a microtitre plate format as outlined in Kleter et al [Am. J. Pathology (1998), 153: 1731-1739].

Optionally the process comprises a detection signal amplification step which is not type-specific.

The invention particularly relates to a process for identification of viral types in a sample, more particularly an HPV type.

The invention also relates to a method for identification of individuals at risk from disease, comprising typing analysis of a sample from the individual using the method of the invention.

The invention further relates to diagnostic kits comprising at least one set of suitable broad spectrum primers in combination with at least one set of type-specific primers, optionally in combination with means appropriate to carry out a type-specific hybridisation reaction.

We have determined that current analysis processes provide only an incomplete picture of the disease risk associated with a given sample. Certain samples may contain for example, viral types with a high association with disease which are not detected at the hybridisation step, even though the hybridisation step contains probes specific for such type detection.

In the case of HPV, for example, we have determined that samples which appear by hybridisation analysis only to contain a type having only a low association with disease also comprise high risk types, despite the presence of specific probes designed to those high risk types. Current testing routines would not identify such high risk types, and such types might not be identified in several samples.

Accordingly the present invention is preferably concerned with typing samples which comprise or may comprise multiple polynucleotide types.

It has been determined that, unless a type of interest has been positively identified by hybridisation, it is not possible to assume that such a type is absent from the sample. This is true even when a specific probe for the type of interest is used during the hybridisation phase. As such, in the present invention, a selective, type-specific amplification step is introduced after any typing process involving a hybridisation step for the detection of specific high risk types not identified by the hybridisation screen. The use of such a specific amplification step allows a more complete determination of the types present, for example a more complete determination of HPV infection, than was previously available.

As for hybridisation-based detection systems, direct sequencing methods suffer from a similar drawback; where there is multiple infection the low level concentration of a type may not be detectable. Accordingly, sequencing alone cannot provide a definitive answer to types present in a sample, due to limited sensitivity. A further type-specific amplification stage is also required.

Without wishing to be constrained by theory, it is thought that competition between polynucleotide types in a sample can lead to certain polynucleotide types being undetected. Competition might occur at a number of different stages in the process. By way of example, in the case of HPV typing using broad spectrum PCR primers, if one HPV type is present in great molar excess over another type it is likely that the minor type will be outcompeted and could remain below the detection limit of the assay. Additionally, since different HPV genotypes may contain slightly different nucleotide sequences at the primer target regions, each genotype will be preferentially amplified by a subset of PCR primers from the available broad-spectrum primer pool. Furthermore, at the signal detection level, the detection of hybridisation between a type-specific probe and its corresponding type-specific polynucleotide target may also be affected where there are multiple types in a sample.

Suitably the present invention comprises a hybridisation/detection step, the result of which is sensitive to competition between polynucleotide types in a sample, for example competition for detection probes or amplification primers.

Suitably the hybridisation/detection step of the present invention is capable of giving a false negative result when used to analyse a sample comprising mixed polynucleotide types.

A type-specific polynucleotide sequence of the present invention represents a specific subset of a broader class of related sequences. In particular a given type is preferably characterised on the basis of sequence and/or hybridisation characteristics, and may be distinguished from members of a broader class on the basis of these parameters.

In the cases of viruses a ‘type’ is preferably a genotype, and identification of a type-specific polynucleotide sequence in a sample is suitably genotype identification. For example, in the case of HPV, viral isolates that display a sequence difference of more than 10% to any previously known type in the L1 gene are classified as different genotypes (Chan et al, Journal of Virology (1995) 69:3074-3083). However, isolates may be further classified, and HPV isolates that differ between 2 and 10% are classified as subtypes, while if the variation is below 2% the isolates are classified as variants. As such, any reference to typing herein includes reference to analysis of types, subtypes and variants, as appropriate.

In the case of single gene mutations, for example, a type may be a specific mutation in a gene associated with disease. In the case of the class of the p53 gene, different mutations in the gene are different p53 types.

As such, a type-specific polynucleotide sequence may be a specific gene sequence or one of a group of closely related sequences such as a type, subtype or variant.

Preferably, the method of the invention is used for the typing of HIV-1, hepatitis C virus (HCV), hepatitis B virus (HBV), cytomegalovirus (CMV), Epstein-Barr virus (EBV), hepatitis D virus (HDV), hepatitis G virus (HGV), Herpes simplex virus (HSV), Human herpes virus (HHV), Varicella-zoster virus (VZV), Helicobacter pylori, Mycobacteria, mycoplasma, rotavirus, and typing of genes associated with disease such as p53. Other preferred typing targets include viruses and genes for which different types correlate with disease development and/or severity. Most preferably the present invention is used in typing of HPV. However, it will be appreciated that the present invention is not technically limited to the source of the nucleic acid, and may be used to type nucleic acid from any gene or virus, as appropriate.

The method of the invention uses a sample, which is suitably biological material, such as a tissue sample, taken from an individual being tested for infection and/or risk of disease. Body fluids such as blood and urine may also be used in the process of the invention. There is generally no requirement to take a sample by any invasive surgical process for DNA typing, and swabs may be used to obtain such samples, for example. Non invasive, non surgical methods for obtaining samples are preferred.

Other suitable methods for obtaining samples from individuals for typing are well known in the art.

In the process of the invention sample polynucleotide/nucleic acid and probe are contacted to allow specific hybridisation, if any, to take place.

The polynucleotide for analysis may be used directly from the sample or, more preferably, after a polynucleotide amplification step (eg PCR) step. In some cases it may be necessary to transcribe RNA to DNA before amplification. In both latter cases the amplified polynucleotide is derived from the sample.

Hybridisation of the polynucleotides may be carried out using any suitable hybridisation method and detection system. Examples of hybridisation systems include conventional dot blot and Southern blots, for example. Preferred is a reverse hybridisation approach, wherein type-specific probes are immobilised on a solid support, and amplified polynucleic acids are labelled in order to detect hybrid formation. Most preferred is the LiPA system described in WO 99/14377, and in Kleter et al, [Journal of Clinical Microbiology (1999), 37(8):2508-2517], the whole contents of which are herein incorporated by reference. In this system the oligonucleotide probes are immobilised on a solid support in parallel lines. However, other reverse hybridisation systems may also be employed, for example, as illustrated in Gravitt et al, [Journal of Clinical Microbiology (1998)36(10):3020-3027] the contents of which are also incorporated by reference.

Preferably the polynucleotide sample is screened simultaneously against multiple probes, under the same conditions of hybridisation and washing.

The type-specific probe of the present invention is suitably a single stranded oligonucleotide designed to hybridise to nucleic acid from a given type, such as a viral genotype, which enables identification of that type in a sample.

Type-specific probe sequences are well known in the art, and any such suitable sequences can be used in the present invention. In the case of HPV, for example, suitable type-specific probes are disclosed in WO9914377. The invention is not restricted to the nature or origin of the type-specific probes that are used in hybridisation step in the present invention.

Type-specific probes may be attached to any suitable solid support, such as microtitre dishes, membranes such as nylon or nitrocellulose, microspheres or chips. Other suitable supports are well known in the art. The type-specific probes may be modified in order to allow fixation or improve hybridisation efficiency. Such probes are thus be used in the context of a solid support to contact polynucleotides of interest in the process of the invention.

There is no restriction on the detection system that may be used to detect a type-specific hybridisation reaction. Either the probe or nucleic acid may be labelled. Preferably the nucleic acid to be screened is labelled. Suitable detection systems include radioactive detection by, for example ³²P and ³⁵S, or non-isotopic detection systems which use, e.g. fluorescence. A suitable non-radioactive detection method is disclosed in EP-A-667918.

The presence of a nucleic type in a sample is confirmed by a positive hybridisation reaction. However, the present invention demonstrates that the absence of a positive result cannot be taken to indicate the absence of a viral type. Accordingly, the process of the invention provides a type-specific amplification step using type-specific primers following the hybridisation screening step.

A primer is suitably a single stranded oligonucleotide sequence which serves to act as a startpoint for the initiation of a primer extension product which is complementary (either 100% or partially) to the nucleic acid strand to be copied. Suitable primers for the amplification of specific DNA types may be designed using methods standard in the art. In addition, type-specific primers for a number of viral and gene types are well documented.

Preferred type-specific primers for amplification of HPV types are described in Baay et al., [Journal of Clinical Microbiology, March (1996), 745-747], Karlsen et al [Journal of Clinical Microbiology September 1996, vol 34, no 9, 2095 - 2100] and Yoshinouchi et al [Journal of Clinical Microbiology. November 1999, Vol 37, N°11, p 3514- 3517.], the sequences of which are incorporated by reference herein. Preferred are any HPV primers specific for genotypes associated with high risk of cervical cancer, such as, but not limited to, HPV 16, 18, 31, 33, 45, 52, 58, 35, 56, and 59.

Type-specific amplification is suitably carried out by the PCR process. The PCR process is well known and documented in the art. The amplification comprises repeated cycles of heat denaturation, annealing of primers to sequences that flank the DNA sequence to be amplified, and extension of the annealed primers with DNA polymerase. The primers hybridise to opposite strands of the target sequence and are oriented such that DNA synthesis by the DNA polymerase proceeds across the region between the primers. Amplification of DNA by the PCR reaction is disclosed in U.S. Pat. Nos. 4,683,202 and 4,683,195, the contents of which are incorporated by reference. In addition, techniques for the analysis of PCR products are standard in the art, such as, for example, sequence analysis or restriction analysis.

Nucleic acid amplification is, however, not limited to PCR and may also be carried out by other suitable methods, such as NASBA (Compton, J (1991) Nature 350:91-92) and LCR (Backman K et al (1989) EP-A 0320 308). Other suitable amplification methods are well known in the art.

Type-specific amplification is suitably followed by a detection step to confirm the presence or absence of an amplimer.

Preferably the type-specific amplification of the present invention is a quantitative process. Quantitative PCR, for example, allows the level of a given nucleic acid type, such as a viral genotype, to be determined and to be correlated with the likelihood of disease and/or disease prevention. In particular, this is important diagnostically when disease risk is increased above a certain threshold of viral load, for example. The correlation between viral load and disease progression, along with quantitative PCR techniques is illustrated in Swan et al. [Journal of Clinical Microbiology, April 1999, 37(4):, 1030-1034] and Josefsson et al. The Lancet, June 2000.355:2189-2193], incorporated herein by reference in respect of such techniques.

The nucleic acid for type-specific amplification is preferably obtained directly from the original sample. Amplification may also be carried out on nucleic acid derived from the sample. For example, where the original sample is RNA, then amplification may be carried out after reverse transcription of the RNA to cDNA. Alternatively, the amplification may be carried out on nucleic acid amplified from the original sample by a broad primer set, for example.

Preferably there is amplification of sample nucleic acid prior to hybridisation screening. Such amplification may be through PCR, or related methods, as discussed above.

Preferably broad spectrum primers are used in any pre-hybridisation step to amplify multiple nucleic acid members of a class of interest, such as HPV DNA. Broad spectrum primers are thus any primers, or groups of primers, which allow amplification of multiple types of nucleic acid from class of related sequences. Broad spectrum primers thus encompass primers which amplify types within a species, subtypes within a type and variants within a subtypes, for example.

In the preferred case of HPV analysis, preferred broad spectrum primers allow for amplification of DNA from at least 30 HPV types, preferably 40 types, more preferably 50 types or even more. Preferably the broad spectrum primers allow for amplification of polynucleotides from different genotypes. Examples of suitable primers are given in Kleter et al [American Journal of Pathology (1998)153(6):1731-1738], and references comprised within, the whole contents and primer sequences of all of which are incorporated by reference. In particular, preferred are the SPF1 and SPF2 primer sets as described in Kleter et al (supra). Other methods and primers for broad spectrum amplification of HPV DNA are given in WO 99/14377, the whole contents of which are incorporated by reference.

The present invention also relates to a process in which there is amplification of nucleic acid pre-hybridisation and/or signal amplification post hybridisation.

The present invention also relates to kits suitable for use in the typing process described above. Kits of the present invention suitably comprise components for viral or gene typing, preferably HPV typing. Preferably kits comprise broad spectrum primers and at least one set of primers which are type-specific. More preferably kits comprise HPV 16 and/or HPV 18 Type-specific PCR primers. Preferably kits also comprise a solid support to which are attached HPV Type-specific probes. Kits may also comprise any necessary hybridisation and wash and detection solutions.

For the avoidance of doubt all publications, including but not limited to patents and patent applications, cited in this specification are herein incorporated by reference as if each individual publication were specifically and individually indicated to be incorporated by reference herein as though fully set forth.

The present invention is now illustrated with respect to the following non-limiting example;

### Examples 1

### Detection of HPV Genotypes

Patients

Eleven patients were selected from a larger group, based on the presence of multiple HPV genotypes comprising HPV16, 18, 31, 33, 35, 51, 52, or 58, as determined by SPF₁₀ PCR and LiPA [Quint W G V et al., Journal of Pathology (2001), 194:154-158]. The selection of samples was based on the detection of those genotypes, for which reliable type-specific PCR primers were available [Baay M F et al. Journal of Clinical Microbiology (1996), 34:745-747; Walboomers J M et al, Journal of Pathology(1999), 189:12-19]. The specificity of each of these type-specific PCRs was reconfirmed by analysis on recombinant plasmids, containing complete genomic DNA from 13 different genotypes, including HPV 16, 18, 31, 33, 35, 39, 51, 52, 56, 59, 58, 59, 66, and 68.

DNA Isolation, PCR and Genotyping

From each patient, a cervical biopsy specimen was obtained. DNA was isolated by guanidinium isothiocyanate treatment and capture onto silica particles [Boom R et al, Journal of. Clinical Microbiology (1990), 28:495-503]. HPV DNA was amplified by SPF₁₀ PCR and amplimers were analysed on the HPV-LiPA as described [Kleter B et al Journal of Clinical Microbiology (1999), 37:2508-2517]. Subsequently, the same DNA isolate was tested by type-specific PCR primer sets of the types that were found positive by LiPA.

Results

The results of the SPF10-LiPA and the type-specific PCRs in biopsy specimens are summarized in Table 1.

In patient 9, HPV 16 and 31 were detected by type-specific PCR in the biopsy specimen, while these types were not identified by SPF₁₀-LiPA. Similarly, in patient 11, SPF₁₀-LiPA identified only HPV 16 in the biopsy specimen, while type-specific PCR detected additional types 51 and 52.

TABLE 1
Identification of HPV genotypes by
SPF₁₀-LiPA and type-specific PCR primers.

| Biopsy | Types found by | Type-specific |
|---|---|---|
| Sample | SPF₁₀-LiPA | PCR |
| 1 | 16, 33 | 16, 33 |
| 2 | 18, 31 | 18 |
| 3 | 18 | 18 |
| 4 | 16, 31 | 16, 31 |
| 5 | 16, 58 | 16, 58 |
| 6 | 16, 18 | 16, 18 |
| 7 | 16, 31, 52 | 16, 31, 52 |
| 8 | 16, 31 | 16, 31 |
| 9 | 18 | 18, 16, 31 |
| 10 | 18 | 18 |
| 11 | 16 | 16, 51, 52 |

1. A process for identification of type-specific polynucleotide sequences in a sample, the process comprising the steps of:
(i) contacting polynucleotides from the sample, or derived from the sample, with a plurality of type-specific probes in the context of a solid support, and detection of any type-specific hybridisation; and
(ii) contacting polynucleotides in the sample, or derived from the sample, with type-specific primers for those types capable of being detected by the hybridisation step in step 1, but not so detected, in a type-specific amplification reaction.

2. A process according to claim 1 additionally comprising a first step of amplifying polynucleotides from the sample using broad spectrum primers and subsequent use of said amplified polynucleotides in step (i)

3. A process according to claim 2, wherein polynucleotide sequences amplified by broad spectrum primers are analysed to confirm the presence of general polynucleotide types of interest prior to hybridisation.

4. A process according to claim 1 wherein the type specific amplification is quantitative.

5. A process according to claim 1, wherein the type specific primers are specific for one of HPV 16, 18, 31, 33, 45, 52, 58, 35, 56, and 59

6. A method for identification of an individual at risk from disease, the method comprising detecting and typing of a polynucleotide correlated with the disease in a sample from the individual using the process according to claim 1.

7. A diagnostic kit for use in the process of claim 1 comprising at least one set of suitable broad spectrum primers in combination with at least one set of type-specific primers.

8. A kit according to claim 7 comprising HPV 16 and/or HPV 18 type-specific PCR primers.

9. A process, method or kit according to claim 1 for the typing of HPV, HIV-1, HCV, HBV, CMV, EBV, HDV, HGV, HSV, HHV, VZV, Helicobacter pylori, Mycobacteria, mycoplasma, rotavirus or gene p53.

10. A process, method or kit according to claim 9 for the typing of HPV.
